(19) United States
(12) Patent Application Publication
(10) Pub. No.: US 2025/0345522 A1
(43) Pub. Date: Nov. 13, 2025

(54) Spacer for an Add-On Device

(71) Applicant: Sanofi, PARIS (FR)

(72) Inventors: Stephan Riedel, Frankfurt (DE); Michael Joest, Obertshausen (DE)

(21) Appl. No.: 18/869,021

(22) PCT Filed: Jun. 7, 2023
(86) PCT No.: PCT/EP2023/065186

§ 371 Date: Nov. 25, 2024

(30) Foreign Application Priority Data
Jun. 9, 2022 (EP) 22315119.2

(51) Int. Cl. A61M 5/24; A61M 5/20

(52) U.S. Cl. CPC A61M 5/24; A61M 5/20; A61M 2005/2073

(57) ABSTRACT

An add-on device of an injection device includes a device body and a movable part. A spacer for the add-on device is detachably fastenable to the add-on device. The movable part is movable relative to the device body along a first direction. The spacer includes a spacer body including an engaging portion to detachably fasten to one of the device body and the movable part. The spacer body is operable to impede a movement of the movable part relative to the device body with regard to the first direction when fastened to the device body or the movable part.

### CROSS REFERENCE TO RELATED APPLICATIONS

The present application is the national stage entry of International Patent Application No. PCT/EP2023/065186, filed on Jun. 7, 2023, and claims priority to Application No. EP 22315119.2, filed on Jun. 9, 2022, the disclosures of which are incorporated herein by reference.

### TECHNICAL FIELD

The present disclosure relates to an add-on device for an injection device. Furthermore, the disclosure relates to a method of attaching an add-on device to an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure should be easy to operate and be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to an injection device. An add-on device typically comprises a sensor, detector, or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

Detaching and reattaching of an add-on device to an injection device may require some training and should not induce an unintended operation of the add-on device.

Some add-on devices comprise a device body, which is configured for a detachable fastening to a dose dial, which may be provided at a proximal end of an elongated injection device, such as a pen-type injection device. Here, the device body of the add-on device can firmly attach to, e.g. frictionally engage with, the dose dial. The add-on device can further comprise a movable part, e.g. forming or constituting a kind of an auxiliary trigger. The movable part may operably engage with a trigger of the injection device when moved along a first direction relative to the device body.

With some add-on devices that comprise a device body and a movable part, it may be intended that the movable part does not move relative to the device body when the add-on device is not attached to an injection device. Also, in the course of mounting or reattaching the add-on device e.g. to a dose dial of an injection device, the movable part should not be moved or depressed relative to the device body. With some add-on devices it may be necessary to conduct a reset operation of the add-on device after having attached or re-attached the add-on device to another injection device.

Some users may face difficulties to comply with these restrictions, e.g. when attempting to attach the add-on device to the injection device. In the course of assembling the add-on device to the injection device a user may inadvertently move the movable part relative to the device body thereby inducing or initiating a recording action or some other function of the add-on device.

### SUMMARY

Aspects of the present disclosure related to add-on devices with enhanced or improved usability, especially in the course of detaching and reattaching the add-on device from an injection device and to another injection device.

The use of certain add-on devices described herein can be simplified. For example, detaching and attaching the add-on device to an injection device can be simplified. User acceptance for such add-on devices can be improved, and such add-on devices can allow for more intuitive handling.

Some aspects of the present disclosure relate to a spacer detachably fastenable to an add-on device. In certain aspects, the present disclosure relates to an injection device equipped with an add-on device and equipped with a spacer for such an add-on device.

In some aspects, there is provided a spacer, which is detachably fastenable to an add-on device of an injection device. The add-on device comprises a device body and a movable part. The movable part is movable relative to the device body along a first direction. The spacer comprises a spacer body. The spacer body comprises an engaging portion to detachably fasten to one of the device body and the movable part. The spacer body is operable to impede or to prevent a movement of the movable part relative to the device body with regards to the first direction when the spacer body is fastened to the device body or to the movable part.

Insofar and when appropriately attached or fastened to the add-on device the spacer serves to inhibit or to impede a movement of the movable part relative to the device body along the first direction. In this way the spacer may effectively serve to prevent an unintended operation or activation of the add-on device, e.g. in the course of assembling the add-on device to the injection device. Insofar, the spacer comprises or constitutes a stopper, e.g. a mechanical stopper, which is operable to block a movement of the movable part relative to the device body.

The spacer may be attachable to the add-on device before detaching the add-on device from an injection device and/or before attaching the add-on device to the injection device. While the add-on device is detached from the injection device the spacer may remain fastened to at least one of the device body and the movable part, thereby preventing a relative movement of the device body and the movable part with regards to the first direction. In the course or during assembly of the add-on device to the same or to another injection device the spacer effectively prevents a relative movement of the movable part relative to the device body at least with regard to the first direction, which may by a longitudinal direction.

In this way, the process of (re-) assembling the add-on device to the injection device, e.g. the process of attaching the device body e.g. to a dose dial of the injection device, can be simplified. Here, the user may also apply a mounting force to the movable part, which force will not lead to a relative motion between the movable part and the device body. Rather, due to the spacer, e.g. effectively located between the movable part and the device body a respective mounting force, e.g. applied by a user not only onto the device body but also on the movable part, may be transferred via the spacer from the movable part to the device body. In effect and by way of the spacer a relative movement of the movable part and the device body with regards to the first direction can be effectively impeded. An unintended activation or operation of the add-on device during mounting or fastening the add-on device to the injection device can be effectively prevented.

After having properly attached to the add-on device to the injection device the spacer may be detached from the add-on device thereby enabling an intended movement of the movable part relative to the device body along the first direction.

By way of the spacer an unintended operation or actuation of the add-on device can be effectively prevented. Insofar, conducting of a reset operation of the add-on device after attaching the add-on device to another injection device may become superfluous. In this way, an overall handling of the add-on device can be simplified and user satisfaction can be enhanced. According to a further example the engaging portion of the spacer comprises a clasp or clip to at least partially embrace a sidewall of the movable part or to at least partially embrace a sidewall of the device body.

With some examples the device body is of elongated and/or tubular shape. Here, a longitudinal axis may extend along the first direction. Accordingly, the movable part may be movable along the longitudinal direction relative to the device of body, especially for inducing a dose injection procedure when the add-on device is correctly attached or fastened to the injection device. With further examples the engaging portion comprises a clip or the clasp of the engaging portion forms or constitutes a fastening clip. This way, the engaging portion serves to provide a positive or form-fitting engagement between the spacer body and at least one of the sidewalls of the movable part and the device body.

With some examples the clasp or clip of the engaging portion is configured to enter or to at least partially fill a longitudinal gap, hence, a gap with a gap size along the first direction. Here, the longitudinal width or thickness of the engaging portion may correspond to the longitudinal gap size so as to fill or occupy the respective gap. When arranged inside the gap between the device body and the movable part the engaging portion and hence the spacer body acts as a blocking element or a stop element that effectively impedes a respective longitudinal motion of the movable part relative to the device body, which motion would otherwise induce a size reduction of the gap with regard to the longitudinal direction.

With further examples it is conceivable that the engaging portion frictionally and/or positively engages with the sidewall of one of the movable part and the device body and wherein the engaging portion is configured to abut with an end face of the other one of the movable part and the device body, wherein the end face faces towards the first direction.

In this way and when the engaging portion is fixed to the sidewall of one of the device body and the movable part with respect to the first direction a likewise blocking effect can be provided by the spacer when attached or fastened to one of the movable part and the device body.

With some examples the clasp or clip of the spacer or spacer body is configured to at least partially embrace the sidewall of the movable part. In some examples the clasp or clip of the spacer or spacer body is configured to at least partially embrace a sidewall of the device body. In further examples, the clasp or clip of the spacer or spacer body is configured to at least partially embrace both, the sidewall of the device body and the sidewall of the spacer body.

In some examples, the clasp or clip of the spacer or spacer body may comprise an extent along the first direction that is larger than the gap size along the first direction. This way, the clasp or clip of the spacer or spacer body may simultaneously engage with both, at least a portion of the sidewall of the device body and at least a portion of the sidewall of the movable part. Such a configuration is of particular use when the transverse or radial extent of the sidewall of the movable part is substantially identical to the transverse or radial extent of the sidewall of the spacer body.

According to a further example the engaging portion of the spacer body comprises an insert opening to receive at least one of the movable part and the device body. The insert opening of the engaging portion may be sized to receive the sidewall of the movable part or to receive the sidewall of the device body. Both, the sidewall of the movable part and the sidewall of the device body may be of substantially tubular shape. The insert opening of the engaging portion may comprise a circumferential width or size with regards to the circumference of the sidewall of the device body or sidewall of the movable part that is at least slightly smaller than the diameter of the sidewall of the respective device body or movable part. In this way, there can be provided a positive engagement of the engaging portion and at least one of the movable part and the device body.

With some examples the insert opening is provided by the clasp or clip. The clasp or clip of the engaging portion may be sized and configured to embrace at least a portion of the circumference of the sidewall of the device body or movable part. Typically, the clasp or clip is configured and sized to embrace a portion of the sidewall of the movable part or device body that is larger than or at least equal to half of the circumference of the respective sidewall. In this way, there can be provided a positive fit between the clasp or clip and at least one of the sidewall of the movable part or device body. Such a clasp or clip connection provides a mounting or fastening as well as demounting or detaching of the spacer in radial direction with regards to the tubular shape of the sidewall of the movable part or device body.

According to a further example the engaging portion comprises a first arm portion and a second arm portion. The first and the second arm portions may extend diametrically opposite to each other so as to confine a receptacle sized to receive the sidewall of the movable part or the sidewall of the device body. The first and the second arm portions may be symmetrically shaped. They may be sized and configured to embrace a portion of the outside circumference of the sidewall of at least one of the movable part and the device body.

The first arm portion may comprise a free end facing towards a free end of the second arm portion. Opposite their free ends the first and the second arm portions may be mutually connected. The first arm portion and the second arm portion may be integrally formed with the engaging portion. Insofar an end section of the first arm portion facing away the free end of the first arm portion may be integrally formed or may merge with an end section of the second arm portion facing away the free end of the second arm portion.

With a further example the spacer is of planar shape. The spacer may be of disc-like shape. The first arm portion and the second arm portion may extend in a common plane. This way, the spacer can be designed in a rather compact and space saving way. Due to its shape the way of how the spacer and/or its engaging portion can be attached to the movable part or to the device body is rather self-explaining and intuitive.

According to a further example the insert opening of the engaging portion is formed between the free end section of the first arm portion and the free end section of the second arm portion. The circumferential distance between the free end section of the first arm portion and the free end section of the second arm portion is typically less or smaller than half of the circumference of the sidewall of the movable part or of the device body, to which sidewall the spacer is configured to become fastened.

The free end sections of the first arm portion and the second arm portion may face towards each other. They may define the insert opening and hence the circumferential width of the insert opening. With some examples it may be the free end sections of the first arm portion and the second arm portion that engage with a respective portion of the sidewall of at least one of the device body and the movable part. By applying a radially inwardly directed pressure to the sidewall of one of the device body or movable part the free end section may become subject to an evasive movement thereby increasing the distance between the free end sections to such an extent that the sidewall of one of the device body or movable part may snap into the receptacle of the engaging portion.

With some examples the free end section of the first arm portion and the second arm portion may protrude inwardly and hence towards each other so as to engage with a complementary shaped recess provided in the respective sidewall of the device body or movable part. In this way there can be provided a positive-fitting between the arm portions of the engaging portion and a recessed structure on the outside of the sidewall of at least one of the movable part and the device body. This way, the engaging portion and hence the entire spacer can be fixed to the sidewall of one of the device body and the movable part with regards to the first direction. When the spacer or at least a portion of the spacer body extends radially outwardly from the engaging portion, such a radially outwardly extending portion may get in longitudinal movement-impeding abutment with the other one of the device body and the movable part.

According to a further example the first arm portion and the second arm portion are of arcuate shape. An inside edge or inside sidewall of the first arm portion faces towards an inside edge or inside sidewall of the second arm portion. The first and the second arm portions may form or constitute a kind of a semicircular receptacle to embrace or to clasp around at least a portion of the sidewall of the movable part or of the device body.

The shape and/or of the contour of the inside edge or inside sidewall of the arm portion may correspond to an outside contour or outside shape of the sidewall of the device body or movable part. This way, there can be provided a rather firm and stable fixing between the engaging portion and the movable part or device body.

According to a further example at least one of the first arm portion and the second arm portion and hence at least a portion of the engaging portion is elastically deformable to increase a distance between the first arm portion and the second arm portion. Typically, and by an elastic defamation, e.g. in the plane of the planar-shaped spacer body, the size of the insert opening, e.g. formed between the free end sections of the first and second arm portions, can be at least temporally increased so as to receive the sidewall of the movable part or the sidewall of the device body. An elastic deformation of at least one of the first arm portion and the second arm portion can be easily achieved by manufacturing the engaging portion and hence the spacer body from an elastically deformable material.

With some examples the spacer body or at least the engaging portion thereof is made of an elastically deformable plastic material. The spacer body may comprise a thermoplastic material. The spacer or spacer body may comprise an injection molded plastic material. By implementing the spacer or spacer body as a plastic component, e.g. as an injection molded plastic component, the spacer may be producible at low or moderate costs.

According to a further example the spacer body is of planar shape and/or of disc-like shape. A planar- or disc-shaped spacer body is rather easy and intuitive to use. A thickness of the spacer body or of the engaging portion as seen along a surface normal of the planar or disc-shaped spacer body may be adapted to or may conform a recessed structure of the add-on device. In this way the recessed structure provided on an outside surface of the sidewall of the add-on device can be effectively occupied by the spacer body. The size of the recessed structure along the first direction may match with the respective thickness of the spacer body. Insofar and when correctly fastened or attached to the add-on device the spacer body may occupy the recessed structure at least with regards to the first direction thereby effectively impeding a movement between the movable part and the device body, which movement is typically accompanied by a reduction of the size of the recessed structure along the first direction.

According to a further example a thickness of at least one of the spacer body and the engaging portion is adapted to or conforms to a recessed structure provided on an outside surface of the add-on device. In some examples, it may be also the thickness of the clasp that is adapted to or conforms to the recessed structure.

In some examples, the thickness or extent of the spacer body may be uniform across the plane of the spacer body. In other examples, the thickness of the spacer body may vary across the plane of the spacer body.

According to another example a size of the recessed structure of the add-on device along the first direction matches with the thickness of at least one of the spacer body and the engaging portion of the spacer body. In this way, the spacer body and/or its engaging portion may enter the recessed structure and may effectively occupy at least a circumferential portion of the recessed structure as seen along the first direction.

According to another example and when the spacer is correctly fastened or attached to the add-on device, at least one of the spacer body or the engaging portion at least in sections (e.g., at least partially) occupies the recessed structure of the add-on device, thereby impeding a movement of the movable part relative to the device body. In effect, that portion of the spacer body or engaging portion that enters the recessed structure of the add-on device is operable to impede or to block a movement of the movable part relative to the device body along the first direction, which movement would lead to a reduction of the size of the recessed structure along this first direction.

According to a further example the spacer body comprises a gripping portion projecting from the engaging portion. Typically, the gripping portion project outwardly from the engaging portion. With some examples and when the engaging portion comprises a receptacle sized to receive a portion of the outer circumference of a sidewall of the device body or of the movable part, the gripping portion protrudes outwardly from the receptacle. In this way and when the engaging portion is attached to e.g. a tubular-shaped sidewall of the movable part or device body the gripping portion projects radially outwardly. The gripping portion may comprise a gripping flange. It may be located diametrically opposite to the insert opening of the engaging portion. With further examples the gripping portion may be located substantially midway between the first arm portion and the second arm portion. The gripping portion facilitates handling of the spacer by a user. A user may grip the spacer body by and through the gripping portion for fastening the spacer to the add-on device as well as for detaching the spacer from the add-on device.

The gripping portion may comprise or constitute an outwardly protruding or radially outwardly extending gripping tab. Typically, the gripping portion may be grippable by an index finger and a thumb of a user.

With a further example the gripping portion comprises at least one or numerous gripping ribs. The gripping ribs may protrude along the first direction from the e.g. planar-shaped gripping portion. Typically, the engaging portion and the gripping portion are located adjacently next to each other in the plane of the spacer body. The gripping portion is typically located in the same plane as the first and second arm portion.

The gripping ribs may protrude from the plane of the planar-shaped spacer body. The gripping ribs may extend parallel to each other. With some examples the gripping ribs extend in tangential or circumferential direction with regards to a circular or semicircular shape of the receptacle formed by the clasp or clip of the engaging portion. This way, a friction fit between fingers of a user and the gripping portion of the spacer can be enhanced.

According to a further example the spacer comprises a through recess extending through the spacer body. The through recess may be located in the engaging portion or in the gripping portion. The through recess may serve as a fastening structure, e.g. for a line or leash. This way the spacer can be connected to a line or leash when not fastened to the add-on device. The line or leash can be fastened elsewhere to the add-on device or to the injection device, e.g. to a pocket clip of the injection device. When not in use, the spacer can be kept loose-secured with the add-on device or the injection device even when not fastened to the body or movable part.

With a further example the spacer body comprises a proximal face and a distal face. The distal face is configured to abut with a proximal face of the device body. The proximal face is configured to abut with a distal face of the movable part. The proximal face and the distal face of the spacer body are opposite faces of the spacer body. They may face in opposite directions. The proximal face as well as the distal face may be both of planar or disc-like shape. The respective faces extend parallel to each other and the spacer may comprise a substantially constant thickness as seen along the first direction.

The proximal face of the device body and the distal face of the movable part of the add-on device may confine a recessed structure on the outside of the add-on device. When correctly assembled to the add-on device the respective recesses structure between the distal face of the movable part and the proximal face of the device body can be at least partially or even completely occupied by the spacer body thereby inhibiting a decrease of the size of recessed structure if a user should attempt to move the movable part relative to the device body along the first direction.

Here, and when the movable part is slidably supported relative to the device body it is sufficient when only a circumferential portion of the recessed structure between the movable part and the device body is occupied by a respective portion of the spacer. As long as the spacer is fastened to at least one of the movable part and the device body, a movement of the movable part relative to the device body along the first direction is effectively blocked and impeded.

According to a further example the spacer body comprises a thickness, which is less than or equal to a longitudinal distance between the proximal face of the device body and the distal face of the movable part. With the spacer body comprising a uniform thickness there can be provided a rather homogeneous abutment between the spacer body, the distal face of the movable part and the proximal face of the device body. This way, there can be provided a rather stable and robust abutment between the oppositely directed faces of the device body and the movable part and hence a movement impeding function of the spacer when appropriately attached to one of the device body and the movable part.

In certain aspects, an add-on device is detachably connected to an injection device. The add-on device comprises a device body and a movable part. The movable part is movable relative to the device body at least along a first direction. The add-on device further comprises a spacer as described above. The spacer is detachably fastenable to one of the device body and the movable part. The spacer is configured to impede a movement of the movable part relative to the device body at least with regards to the first direction.

Since the add-on device is equipped with a spacer as described above, all features, effects and benefits described above in connection with the spacer equally apply to the add-on device; and vice versa.

With some examples the movable part is movable relative to the device body along the first direction to induce a dose injection procedure when the add-on device is correctly attached to the injection device. Insofar, the movable part may serve as an auxiliary trigger, which is operably engageable with a trigger of the injection device when the add-on device is attached to the injection device.

The movable part may be configured for detachably connecting to a dose dial of the injection device. The movable part and the device body may be rotatable relative to each other, e.g. along a second direction. The spacer may be configured to exclusively impede a movement along the first direction while allowing and/or supporting a movement along the second direction, which may be a rotation with the longitudinal axis as an axis of rotation.

Typically and with regards to a tubular shape of e.g. the device body or movable part the first direction extends parallel to the cylindrical or longitudinal tubular axis of the device body or movable part. The second direction may extend in circumferential direction with regard to the tubular shape of the device body or movable part. The movable part may be longitudinally slidable relative to the device body along the first direction. It may be rotatable along the second direction relative to the device body with a longitudinal axis of the device body as an axis of rotation.

Insofar, the spacer may exclusively serve to impede a longitudinal movement of the movable part relative to the device body so as to impede an actuation of the add-on device, which typically comes along with the dispensing operation of an injection device when the add-on device is correctly attached to the injection device.

With the spacer fastened to the add-on device a user may even apply a pressure or a mounting force to the movable part, which pressure or mounting force is effectively transferred via the spacer to the device body without substantially moving the movable part relative to the device body at least with regard to the first, i.e. the longitudinal direction. In this way, the spacer facilitates and simplifies a mounting of the add-on device to the injection device.

With a further example the device body of the add-on device comprises a receptacle, which is configured to receive a dose dial of the injection device. Typically, the receptacle is configured to frictionally engage with the dose dial of the injection device. The dose dial of the injection device may be of substantially tubular or sleeve like shape. The receptacle of the device body may comprise one or several elastic fastening ribs to provide a slip free frictional engagement between an inside of the receptacle and an outside of the dose dial of the injection device.

With further examples the outside of the dose dial and/or the inside of the receptacle may comprise one or several radial recesses or protrusions so as to provide a slip-free form fitting or positive fitting between the dose dial and the receptacle of the device body.

The movable part may protrude proximally and hence in proximal longitudinal direction from the device body. The movable part may comprise a button that is depressible by e.g. a thumb of a user. The movable part may comprise a stem extending longitudinally into and/or through the receptacle of the device body so as to mechanically engage with a trigger of the injection device when the add-on device is correctly attached to the injection device. Since the movable part is movable at least in longitudinal direction with regard to the device body the movable part may be operable to induce a distally directed actuation force onto the trigger of the injection device when the device body is fastened to the dose dial. In this way, the movable part may act as an auxiliary trigger.

According to a further example the movable part is operably engageable with a trigger of the injection device. The trigger of the injection device is depressible or movable relative to the dose dial with regard to the first direction. When the add-on device is attached to the injection device the trigger is depressible or movable relative to the dose dial with regard to the first direction by moving the movable part relative to the device body along the first direction. Since the device body is fixed to the dose dial a movement of the movable part along the first direction relative to the device body serves to induce a respective movement of the trigger relative to the dose dial.

Insofar, the general operability of the injection device, namely to e.g. rotate a dose dial and to depress a trigger for setting and/or injecting of a dose of a medicament can be maintained even when the add-on device is attached to the dose dial and/or trigger of the injection device, thereby entirely covering the dose dial and the trigger.

According to a further example the add-on device comprises an electronic module. The electronic module is arranged inside the device body or inside the movable part. The electronic module comprises a processor, a memory and a transceiver. The electronic module is operable to detect and/or to record injection-related information of the injection device. The electronic module may be further equipped with a detector and/or a sensor to detect a date and/or a time at which a user operates the injection device. With some examples the detector or sensor is operable to quantitatively measure a size of a dose actually set or injected.

With further examples the electronic module comprises a transceiver operable to establish a communication link with an external electronic device, such as a smartwatch, a smart phone or a tablet computer. The electronic module may be further configured or operable to transmit recorded injection-related data to the external electronic device for further data analysis.

According to a further example the add-on device comprises a recessed structure on an outside of a sidewall of the add-on device. A size of the recessed structure along the first direction may match with a respective thickness of at least one of the clasp, the engaging portion and the spacer body of the spacer. Typically, the recessed structure is provided between the device body and the movable part. The recessed structure may comprise a circumferentially extending recessed structure. It may be of annular or oval shape and may comprise a closed annular geometry, e.g. between the movable part and the device body.

In some examples the recessed structure may comprise an annular groove, e.g. in one of a sidewall of the device body and the movable part. In some examples the recessed structure may be provided as an annular groove confined by an annular face of the movable part and an annular face of the device body. In some examples the recessed structure is confined along the first direction by a proximally facing annular face of the device body and by a distally facing annular face of the movable part. Here, respective faces of the movable part and of the device body may comprise or may constitute abutment faces to engage or to abut with the spacer body, the clasp or the engaging portion of the spacer.

According to a further example, the recessed structure on the outside of the add-on device is confined by a proximal face of the device body and a distal face of the movable part. Typically, the distal face of the movable part may be offset towards the proximal direction from the proximal face of the device body. For operating the add-on device, it may be desired or required to move the movable part in distal direction, thereby reducing the size of the recessed structure along the first direction. When and as long as the recessed structure is occupied by the spacer, such a movement is effectively impeded or blocked by the spacer.

According to further aspects, an injection device can operate to set and inject a dose of a medicament. The injection device comprises a housing to accommodate a medicament container. The medicament container e.g. contains a liquid injectable medicament. The injection device further comprises a drive mechanism operably engageable with the medicament container for dispensing or for injecting of the dose of the medicament. The injection device further comprises a dose dial operable by a user for setting of the dose and further comprises a trigger depressible by the user for injecting or dispensing of the dose.

The dose dial and the trigger may both belong to the drive mechanism. With some examples the drive mechanism comprises a piston rod operable to exert a longitudinally directed pressure onto a stopper of the medicament container in order to expel the dose of the medicament from a distally located outlet of the medicament container. With some examples the outlet of the medicament container may be easily connected with an injection needle.

The injection device is further equipped with an add-on device as described above. The add-on device is attachable to at least one of the dose dial and the trigger. Typically, the device body of the add-on device is fastenable or attachable to the dose dial and the movable part of the add-on device is operably engageable with the trigger of the injection device when the add-on device is attached to the injection device.

Insofar, all features and effects as described above in connection with the spacer and the add-on device equally apply to the injection device; and vice versa.

Some aspects of the present disclosure can encompass a method of attaching and/or fastening and add-on device to an injection device. Here, the method makes use of a spacer as described above. In a first step a spacer as described above is attached to the device. With the spacer attached to the add-on device the add-on device is then attached or mounted to the injection device. After attaching or mounting the add-on device to the injection device the spacer is removed. It is hence detached from at least one of the device body or movable part of the device, thereby enabling a movement of the movable part relative to the device body with regards to the first direction.

The method is particularly dedicated to be conducted with a spacer, an add-on device and an injection device as described above. Insofar, all effects, features and benefits as described above in connection with the spacer, the add-on device and the injection device equally apply to the method of attaching or fastening the add-on device to the injection device; and vice versa.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of AIport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 schematically illustrates an add-on device for attaching to the proximal end of the injection device,

FIG. 3 schematically illustrates a cross-section through the assembly of the injection device and the add-on device with the movable part in an initial configuration,

FIG. 4 shows the assembly of FIG. 3 with the movable part depressed,

FIG. 5 is illustrative of the add-on device equipped with the spacer,

FIG. 6 schematically illustrates a spacer for detachably fastening to the add-on device,

FIG. 7 shows another example of an add-on device in an initial configuration,

FIG. 8 shows the example of FIG. 7 with the movable part depressed,

FIG. 9 shows an example of the spacer attached to the add-on device of FIG. 7,

FIG. 10 shows another example of a spacer,

FIG. 11 shows a further example of a spacer attached to an add-on device,

FIG. 12 shows a flowchart of a method of fastening an add-on device to an injection device,

FIG. 13 schematically illustrates a typical scenario of use of the injection device,

FIG. 14 shows a block diagram of an example of the add-on device,

FIG. 15 shows a schematic illustration of the add-on device mounted on the injection device before reaching a final assembly configuration,

FIG. 16 shows the assembly of FIG. 15 with the add-on device is its final assembly configuration, and

FIG. 17 shows the assembly according to FIG. 16 after removal of the spacer.

### DETAILED DESCRIPTION

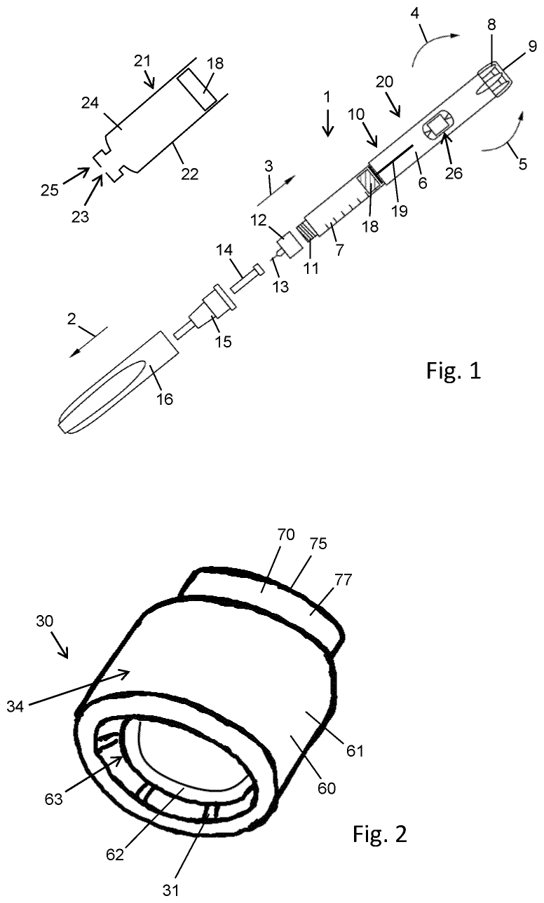

FIG. 1 shows an example of a drug delivery device 1, which is implemented as a handheld injection device. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a cartridge holder 7. The body 6 may be sized and configured to accommodate a drive mechanism 20. The cartridge holder 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the cartridge holder 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force sufficient to move the piston rod 19 and hence the piston 18 in distal direction 2. With other examples, the drive mechanism comprises a mechanical energy storage configured to provide at least a portion of the dispensing force. Examples of drive mechanism can be found e.g. in WO2004/078241 A1, WO 2014/033197 A1 or WO 2014/033195 A1 the entirety of which are herein incorporated by reference.

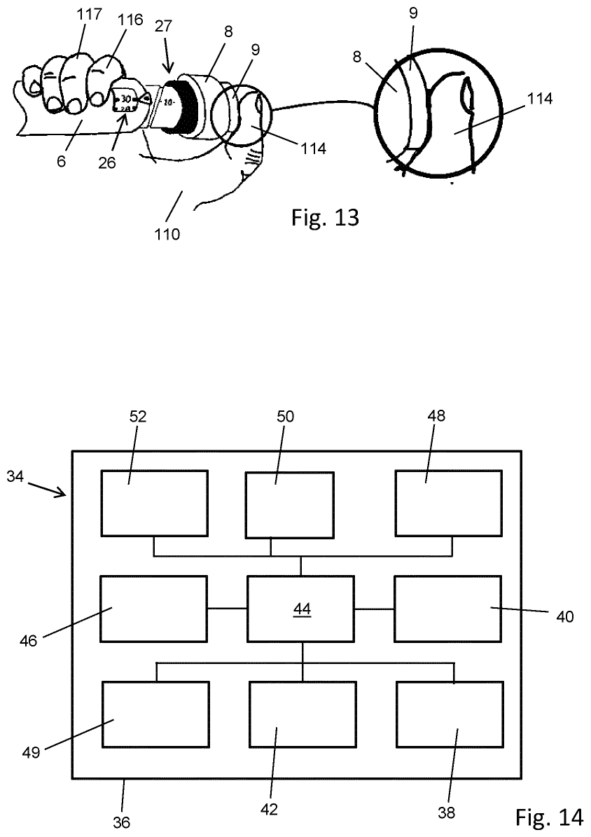

With some examples, as e.g. described or shown in FIG. 13 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of the user's hand 110 to exert a distally directed pressure onto the trigger 9 thereby urging the dial extension 27 in distal direction 2 during a dose injection procedure. During the injection procedure the injection device 1 may be firmly held by the fingers 116, 117 and/or the palm of the hand 110.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the cartridge holder 7 has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the cartridge holder 7. It comprises a double-tipped injection needle 13. A proximal end of the injection needle (not shown) is configured to enter into a through opening at the distal end face of the connector 11 or cartridge holder 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The cartridge holder 7 and hence a portion of the housing 10 is to be received in a protective cap 16, which is detachably connectable to the cartridge holder 7 or body 6.

An add-on device 30 for use with the injection device 1 is illustrated in FIGS. 2-12 and 14. The add-on device 30 comprises a device body 60 of substantially tubular shape. The device body 60 comprises a tubular-shaped sidewall 61. Towards a distal end the device body 60 comprises a receptacle 63. The receptacle 63 is sized to receive a proximal portion of the injection device. Specifically, the receptacle 63 is configured and sized to receive the dose dial 8 as provided at the proximal end of the injection device 1. On an inside surface of the receptacle 63, hence on an inside of the sidewall 61 there are provided numerous fastening ribs 31, e.g. made of an elastic or elastomeric material. The fastening ribs 31 of the receptacle 63 may be configured to form a friction fit with an outside surface of the dose dial 8.

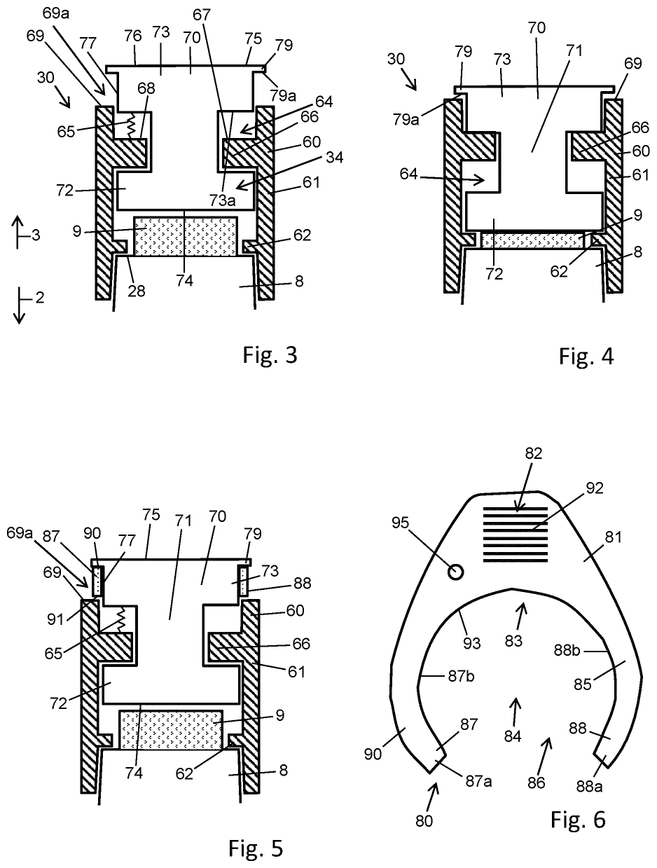

As schematically illustrated in FIGS. 3-5, the outside surface of the dose dial 8 and/or an inside surface of the receptacle 63 may be of somewhat conical shape so as to provide a friction fit and hence a clamping effect between an inside surface of the receptacle 63 and an outside surface of the dose dial 8. This way, there may be provided a press-fitting or form-fitting configuration of the dose dial 8 and the device body 60.

As illustrated in FIGS. 3-5, a final assembly position of the device body 60 with regards to the dose dial 8 has been reached when a radially inwardly extending flange portion 62, delimiting the receptacle 63 towards the proximal direction 3 engages or abuts with a proximal end face 28 of the dose dial 8 of the injection device 1.

The add-on device 30 comprises a movable part 70, which is movable with regards to a first direction, hence with regard to the longitudinal direction relative to the device body 60. The movable part 70 may be also movable relative to the device body 60 along a second direction. The second direction may define a rotational motion of the movable part 70 relative to the device body 60 with a longitudinal axis of the sleeve-shaped device body 60 as an axis of rotation.

With regard to the first direction the movable part 70 is slidably displaceable relative to the device body 60 as can be seen by a comparison of FIGS. 3 and 4. The movable part 70 is movable from an initial position along the first direction in distal direction 2 into a trigger or depressed position. The movable part 70 is also movable from the depressed position back towards the initial position along the longitudinal axis but in proximal direction 3.

In the final assembly position, and when the device body 60 is firmly attached to the dose dial 8 it can be ensured, that the movable part 70, specifically a distal abutment face 74 thereof is located in a well-defined position relative to the trigger 9 of the injection device 1.

Typically, the add-on device 30 is configured for detachable fastening to the proximal end of the dial extension 27. The device body 60 engages with and covers the dose dial 8. The movable part 70 is configured to engage with the trigger 9 or dose button. Insofar, the movable part 70 acts as an auxiliary trigger to transfer a dispensing force, e.g. applied by a thumb 114 of a user to the dial extension 27 and effective to depress the trigger 9 relative to the dose dial 8. Insofar, the typical scenario of use as illustrated and described with regard to FIG. 13 remains substantially unaffected when the add-on device 30 is attached to the dose dial 8 and/or trigger 9.

The movable part 70 of the add-on device 30 comprises a proximal portion 73 protruding in proximal direction 3 from a proximal end or proximal end face 69 of the device body 60. The movable part 70 further comprises a stem 71 connecting the proximal portion 73 with a distal portion 72. The distal portion 72 is located entirely inside the device body 60. The distal portion 72 comprises a distally facing abutment face 74, which is configured to engage with a proximal end face of the trigger 9. Due to the longitudinal sliding displacement of the movable part 70 relative to the device body 60 a user may exert a dispensing force onto a proximally facing end face 76 of the movable part 70 thereby inducing a distally directed motion of the movable part 70 relative to the device body 60.

In an initial configuration as illustrated in FIG. 3 and when applying a distally directed force onto the movable part 70 a respective distally directed movement of the movable part 70 is induced in distal direction 2, thereby transferring a respective force effect onto the trigger 9 by way of which a dose dispensing or dose injection action of the injection device can be triggered or conducted.

During such a dispensing action the dose dial 8 may be subject to a dose decrementing rotation along the dose decrementing direction 5 while the trigger 9 and/or the movable part 70 being in longitudinal abutment with the trigger 9 remain in a non-rotative state.

Due to the frictional engagement between the device body 60 and the dose dial 8 the device body 60 may be subject to a respective rotation in the dose decrementing direction 5 as well and may thus rotate relative to the movable part 70. Insofar and during the stage of dose dispensing there may occur a rotation of the device body 60 relative to the movable part 70. Such a rotation and hence the degree of rotation may be indicative of the size of a dose currently dispensed or injected.

The add-on device 30 may comprise an electronic module 34 as schematically illustrated in FIG. 14. The electronic module 34 may be operable to detect the movement of the movable part 70 along the first direction 2, 3 as well as to detect a movement of the movable part 70 relative to the device body 60 along a second direction, wherein the second direction is one of the dose incrementing direction 4 and the dose decrementing direction 5.

With some examples the device body 60 comprises a proximal receptacle 64 sized to receive the proximal portion 73 of the movable part 70. The add-on device 30 may further comprise a return element 65, e.g. in form of a return spring that is operable to urge the movable part 70 into the initial configuration relative to the device body 60, in which initial configuration the movable part 70 is in a proximal end position. By way of the return element 65 the movable part 70 may automatically return into the initial configuration.

With other examples the add-on device 30 may be void of such a return element 65. Here, a return motion of the movable part 70 into the proximal direction 3 towards and into the initial configuration or position may be induced by a return motion of the trigger 9 relative to the dose dial 8. The injection device 1 and hence the trigger 9 may be depressible in distal direction 2 against the action of a return element, specifically against the action of a return spring or trigger spring (not illustrated). At the end of the dose injection procedure and when releasing the trigger 9 or dose button the trigger 9 or dose button automatically returns into the initial configuration under the effect of the return spring as e.g. illustrated in FIG. 3 or 5.

With the add-on device 30 correctly attached to the dose dial 8, the e.g. spring-induced return motion of the trigger 9 may lead to a respective return motion of the movable part in proximal direction 3 since the distal end face or abutment face 74 of the distal portion 72 of the movable part 70 is in permanent longitudinal abutment or engagement with the trigger 9 when the add-on device 30 is correctly attached or assembled to the proximal end of the injection device 1.

With some examples there is provided a radially inwardly extending projection 66 in the region of the proximal receptacle 64. The projection 66 may provide a proximally facing abutment face 67, which is operable to engage or to abut with a complementary shaped abutment portion or abutment face 73a of the proximal portion 73 of the movable part 70. In this way, a distally directed movement of the movable part 70 relative to the device body 60 can be delimited or blocked.

The projection 66 or some other portion inside the proximal receptacle 64 may be provided with a scale 68 or with a similar rotary encoding structure. The scale 68 may cooperate with a sensor 48, 49, 50 of the electronic module 34 arranged on or inside the movable part 70. The sensor may be implemented as an acceleration sensor 48, as a rotation sensor 49 or as a position sensor 50. The sensor may comprise at least one of an optical sensor, a magnetic sensor and a capacitive sensor arrangement so as to quantitatively measure a degree of rotation and/or a degree of longitudinal movement of the movable part 70 relative to the device body 60. The scale 68 cooperating with the sensor(s) 48, 49, 50 may be optically, magnetically or capacitively encoded.

The movable part 70 may comprise a hollow structure so as to provide construction space to receive or to house the numerous components of the electronic module 34. The distal portion 72 and the proximal portion 73 may be implemented as a single piece. With some examples the distal portion 72 and the proximal portion 73 may be detachably connected. With further examples the proximal portion 73 may be covered by a cover 75, which may be detachable.

Below the cover 75 there may be provided a battery or power source 46, which may be exchangeably arranged inside the hollow interior of the proximal portion 73. The battery may provide an electric energy supply for the electronic module 34.

The electronic module 34 as schematically illustrated in FIG. 14 may comprise a printed circuit board 36. It may further comprise a transceiver 38, a memory 40, a clock 42, a processor 44, a power source 46, an acceleration sensor 48, a rotation sensor 49, a position sensor 50, a signal generator 52 e.g. equipped with light sources. It may also comprise a microphone.

Moreover, a sidewall 77 of the movable part may comprise a window aligned with a respective window in the sidewall 61 of the device body 60. This way, visual signals producible by light sources located inside the movable part 70 can be perceived and visually detected from outside the add-on device 30.

Such light sources may belong to a visual signal generator 52, that is operable to produce or to generate visual signals of different color and/or of variable duration. The windows may be provided with a light pipe or with a light guiding structure. This way, the device body 60 can be protected against ingress of dust or humidity.

As indicated in the block diagram of the electronic module 34 the processor 44 may be mounted on the printed circuit board 36, which may be is fastened or fixed inside the movable part 70 or the device body 60.

In general, the entire electronic module 34 can be mounted on the printed circuit board 36. The electronic module 34 may be configured to communicate with an external electronic device, which may be implemented as a mobile electronic device. It may comprise a smartwatch, a smart phone or a tablet computer. The electronic module 34 comprises a transceiver 38, which is configured to establish or to built-up a communication link between the external electronic device and the electronic module 34. A respective communication link can be realized in a wireless or wired manner.

The electronic module 34 may be configured to exchange data with the external electronic device (not illustrated). Data being indicative of an operation of the injection device and gathered or collected by the sensors 48, 49, 50 may be transmitted to the external electronic device via the transceiver 38. The transceiver 38 may be implemented as a Bluetooth transceiver or as a BLE transceiver. The transceiver 38 may be also implemented as a NFC transceiver. There may be provided numerous transceivers that distinguish from each other with regard to their communication protocol and/or with regard to their spatial range.

The electronic module 34 comprises a memory 40 configured to store a plurality of user interactions with the add-on device. The electronic module 34 further comprises a clock 42 configured to provide each of a plurality of measurement data of the sensors 48, 49, 50 with a time index indicating the point of time and/or date of the detection of the respective operation of the injection device 1.

The electronic module 34 is further equipped with a power source 46 configured to power the processor 44 and the sensors 48, 49, 50. The power source 46 may be implemented as a battery. The acceleration sensor 48 may be configured to detect an injection operation of the injection device 1 and/or to detect or to classify gestures of a user when using the add-on device 30 and/or the injection device 1 provided with the add-on device 30. For example, and when implemented as an autoinjector, the acceleration sensor may detect an acceleration of a needle of the autoinjector upon executing a dispensing or injection procedure.

The position sensor 50 may be operable to detect the position or orientation of a dedicated component of the drive mechanism 20 of the injection device 1. The position sensor may be operable to detect e.g. the position of a last dose nut, of a piston rod 19 or the like component of the drive mechanism 20 being indicative of the amount of medicament located inside the medicament container 21.

Moreover, the electronic module 34 comprises a signal generator 52, which may be implemented as a visual signal indicator comprising at least one light source. Additionally or alternatively, the signal generator 52 may be implemented as a haptic signal generator, e.g. configured to generate a perceivable vibration of the electronic module 34. With further examples the signal generator 52 may comprise an audible signal generator, configured to generate an audible sound.

In addition and with further examples the electronic module 34 may comprise a microphone, by way of which characteristic click noises of the injection device can be detected and processed, thereby allowing to derive or to measure a size of a dose currently set or injected by the injection device 1.

With some examples and when depressed by a user an outwardly facing sidewall 77 of the proximal portion 73 of the movable part 70 may enter into the proximal receptacle 64 as defined by the sidewall 61 of the device body 60. With some examples the proximal portion 73 comprises a radial projection 79, e.g. at the proximal end face 76 that slightly protrudes from the sidewall 77. The distally facing portion of the projection 79 comprises a distal face 79a facing towards the proximal end face 69 of the sidewall 61 of the device body 60.

In a depressed configuration as illustrated in FIG. 4 the distal face 79a may get into abutment with the end face 69, thereby delimiting the distally directed motion of the movable part 70 relative to the device body 60 along the first, e.g. the distal direction 2. With some examples of the add-on device 30 the depression or longitudinal sliding motion of the movable part 70 relative to the device body 60 is automatically detected by at least one of the sensors 48, 49, 50 of the electronic module 34. Such an automated movement detection may trigger a dose injection recording or may induce a particular operation of the electronic module 34.

With some examples and by detecting a depression of the movable part 70 relative to the device body 60 in longitudinal distal direction 2 the electronic module 34 and hence the processor 44 may start to monitor and set or to measure a degree of rotation of the device body 60 relative to the movable part 70 with the longitudinal axis as an axis of rotation. Here, the movement of the rotary encoded scale 68 may be detected and/or monitored by any of the sensors 48, 49, 50.

For detaching and/or attaching the add-on device 30 to the proximal end of an injection device 1, and specifically in order to firmly attach the device body 60 to the dose dial 8 it may be beneficial of not inducing a relative motion between the movable part 70 and the device body 60. In particular, the movable part 70 should not be depressed in distal direction 2 relative to the device body 60 when the add-on device 30 is mounted to the dose dial 8 of the injection device 1.

In order to impede an unintended longitudinal motion, hence a motion along a first direction 2, 3 of the movable part 70 relative to the device body 60 during the process of assembling or attaching the add-on device 30 to the dose dial 8 there is provided a spacer 80 as illustrated in FIGS. 5 and 6.

The spacer 80 comprises a spacer body 81 of planar and/or disc-like shape. The spacer body 81 comprises an engaging portion 83, which is configured to detachably fasten to one of the device body 60 and the movable part 70. When attached to one of the device body 60 and the movable part 70 the spacer 80 effectively blocks and/or impedes a movement of the movable part 70 relative to the device body 60 with regards to the first direction, i.e. with regards to the longitudinal direction 2, 3.

In particular, the spacer 80 and hence the engaging portion 83 is operable to impede a distally directed motion of the movable part 70 relative to the device or body 60. The spacer 80 comprises a clasp 85 comprising a first arm portion 87 and a second arm portion 88. The clasp 85 is configured for a clip-like or snap-fit fastening to the sidewall 77 of the movable part 70. As illustrated in FIGS. 5 and 6, the first and second arm portions 87, 88 enclose at least a major part of the circumference of the sidewall 77 of the movable part 70. This way, a radially recessed structure 69a located between the projection 79 and the proximal end face 69 can be at least partially occupied or filled with the spacer body 81. A user attempting to depress the movable part 70 in distal direction 2 will experience a blocking. Here, the spacer body 81 located between the distal face 79a of the projection 79 and the proximal face 69 of the device body 60 effectively blocks such a distally directed movement. Any distally directed force applied onto the end face 76 and hence onto the movable part 70 will transfer across the spacer 80 onto or towards the proximal end face 69 of the sidewall 61 of the device body 60.

Hence, a user attempting to press the device body 60 in distal direction 2 onto the dose dial 8 may even apply a distally directed force onto the movable part 70, which distal force may an directly transfer to the device body 60 without inducing a longitudinal relative motion between the movable part 70 and the device body 60.

The first and second arm portions 87, 88 each comprise a free end 87a, 88a. The free end 87a, 88a delimit an insert opening 86 in circumferential direction. The insert opening 86 is sized to receive and/or to embrace the sidewall 77 of the movable part 70. In order to attach the spacer 80 to the sidewall 77 the first and second arm portions 87, 88 are elastically deformable to a certain extent so as to increase the insert opening 86 at least temporally to receive the diameter of the tubular-shaped sidewall 77 of the movable part 70, which sidewall 77 protrudes proximally from the proximal end of the sidewall 61 of the device body 60. As long as the spacer 80 is attached to the movable part 70, the movable part 70 is effectively blocked to become displaced in distal direction 2 relative to the device body 60.

The spacer 80 and the spacer body 81 is of substantially planar shape. The arm portions 87, 88 define a receptacle 84 of arcuate or semicircular shape. The inner circumference of the receptacle 84, typically formed by inside edges 87b, 88b or inside sidewalls of the first and second arm portions 87, 88 comprises a circumferential length which is at least slightly larger than half of the circumference of the sidewall 77. In this way, there can be provided a form fitting radial engagement between the engaging portion 83 and the sidewall 77 of the movable part 70.

The circumferential extent of the receptacle 84 is larger than 180° and typically smaller than 300°. More typically, is larger than 200° and smaller than 270° with respect to the circumference of the sidewall 77. The circumferential extent of the receptacle 84 is typically defined by the inner circumference extending from the free end 87a of the first arm portion 87 and the free end 88a of the second arm portion 88. An inner edge 93 of the receptacle 84 of the engaging portion 83 may be of somewhat semicircular or of partial circular structure. The inner edge 93 formed by the first and second arm portions 87, 88 may closely match with the outside structure of the sidewall 77 of the movable part 70.

Due to the elasticity of the arm portions 87, 88 and/or the elasticity of the material the spacer body 81 is made of the insert opening 86 may slightly widen in circumferential direction in an attempt when the insert opening 86 is pushed in radial direction onto the sidewall 77 of the movable part 70. This way and when the largest diameter of the sidewall 77 passes the insert opening 86 the arm portions 87, 88 may relax or return into their initial configuration, thus forming a snap fit connection.

The spacer 80 further comprises a gripping portion 82 protruding radially from the engaging portion 83. The gripping portion 82 comprises or constitutes a gripping tab, which facilitates a manual handling and/or a manual gripping of the spacer 80. When attached to the movable part 70 the gripping portion 82 protrudes radially outwardly from the movable part 70 and is easily grippable by e.g. the fingers of a user for detaching the spacer from the add-on device 30.

As further indicated in FIGS. 3-11 the disc-shaped or planar shaped spacer body 81 comprises a proximal face 90 facing towards the proximal direction 3 and further comprises a distal face 91 facing towards the distal direction 2 when attached to the add-on device 30. The proximal face 90 and the distal face 91 extend substantially parallel to each other. In the region of the gripping portion 82 there may be provided numerous gripping ribs 92. The gripping ribs 92 may be equally provided on the proximal face 90 as well as on the distal face 91. The gripping ribs 92 slightly protrude from the plane of the spacer body 81 and facilitate a slip free and hence precise gripping of the gripping portion 82 by the fingers of a user.

As a further option the spacer body 81 may comprise a through recess 95. The through recess may provide a fastening of a line or leash by way of which the spacer 80 can be movably attached to e.g. the protective cap 16 and/or to any other part of the injection device 1 or add-on device 30 when the engaging portion 83 is released from the movable part 70.

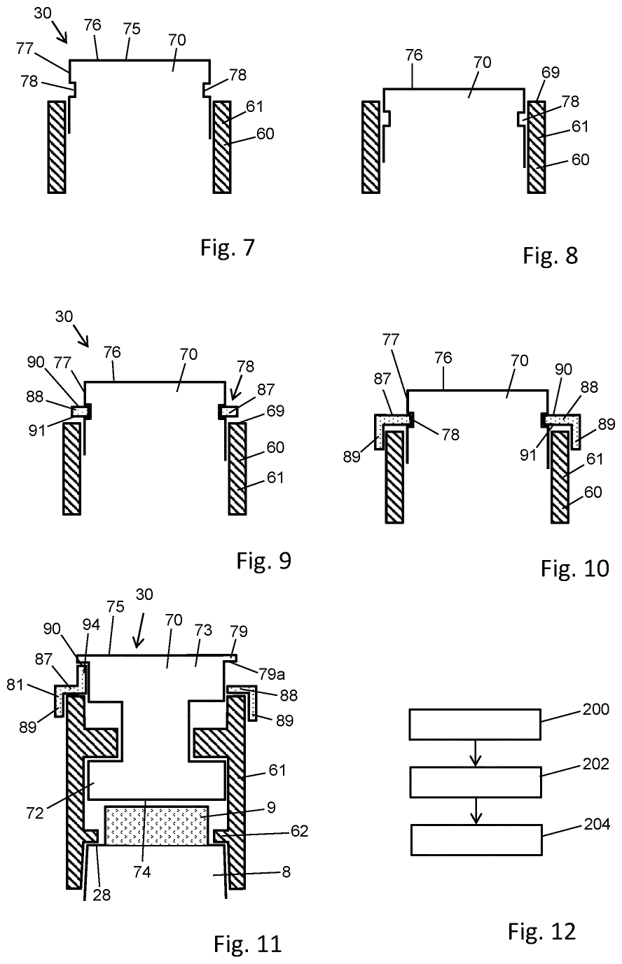

In FIGS. 7-9 there is illustrated a further example of an add-on device 30. There, the movable part 70 is void of a radially outwardly extending projection 79. Rather the sidewall 77 of the movable part 70 comprises a groove 78, which is sized to receive the engaging portion 83 of the spacer 80. The configuration as illustrated in FIG. 7 corresponds to the initial configuration of the add-on device 30, namely when the movable part 70 is not yet depressed by a user. In the illustration of FIG. 8 the movable part 70 is actually depressed by a user to induce a dose dispensing or dose injection procedure. With the spacer 80 attached to the movable part 70 and hence when the clasp of the spacer 80 is engaged with the groove 78 the spacer 80 and hence at least one of the arm portions 87, 88 and the gripping portion 82 radially protrudes from the outside surface of the sidewall 77 and effectively provides a stop to engage with the proximal end face 69 of the sidewall 61 of the add-on device 30.

A user attempting to depress the movable part 70 as illustrated in FIG. 9 will be hindered to do so because the spacer 80 is in form fitting longitudinal engagement with the movable part 70 and blocks a distally directed motion when it's the distal face 91 abuts the proximal end face 69 of the sidewall 61 of the device body 60.

With another example as illustrated in FIG. 10 the spacer 80 comprises a sidewall 89, e.g. protruding in distal direction from the planar-shaped arm portions 87, 88. Here, an inside surface of the sidewall 89 is configured to engage with an outside of the sidewall 61 of the device body 60. Here, the spacer 80 is e.g. detachably connectable to the proximal end of the sidewall 61 of the device body 60 and engages with its radially inwardly extending arm portions 87, 88 with the groove 78 as provided in the outside surface of the sidewall 77 of the movable part 70.

In contrast to the example as illustrated in FIGS. 3-5 the spacer 80 as illustrated in FIG. 10 may positively engage with the recessed structure, e.g. with the groove 78 as provided on or in the sidewall 77 of the movable part 70 and may block a distally directed motion of the movable part 70 relative to the sidewall 61 of the device body 60.

With a further example and as illustrated in FIG. 10 the spacer 80 may be detachably connectable with both, the device body 60 and the movable part 70. The sidewall 89 of the spacer 80 may be in frictional or in positive engagement with the sidewall 61. The spacer 80 or the spacer body 81 may be further in frictional and/or positive engagement with the sidewall 77 of the movable part 70.

With a further example as shown in FIG. 11 the spacer 80 is detachably connectable or detachably fastenable to the proximal end of the sidewall 61 of the device body 60. Here, the spacer body 81 may comprise a proximally extending projection 94 to abut with the distal face 79a of the projection 79 of the proximal portion 73 of the movable part 70. As long as the spacer 80 is attached to the device body 60 a distally directed movement of the movable part 70 relative to the device body 60 is effectively blocked. Operation of the add-on device 30 and hence depressing of the movable part 70 relative to the device body may include the removal of the spacer 80 from the device body 60.

The method of attaching or fastening the add-on device 30 to the injection device is illustrated by the flowchart of FIG. 12. There, and in a first step 200 it is intended to attach a spacer 80 as described above to the add-on device 30. Here, the spacer 80 is either attached to the movable part 70 and/or is attached to the device body 60. In step 202 and when the spacer 80 is correctly attached to the add-on device 30 the add-on device 30 with the spacer 80 attached thereto is connected or attached to the proximal end of the injection device 1. Here, the device body 60 with its receptacle 63 is e.g. press fitted onto the proximal end of the dose dial 8 until the flange portion 62 as illustrated in FIGS. 3-5 gets into longitudinal or distal abutment with the end face 28 of the dose dial 8.

During this fastening or mounting operation the user may even apply a distally directed pressure onto the movable part 70. Any distally directed pressure applied to the movable part 70 can or will be transferred via the spacer 80 onto the device body 60 whilst avoiding a relative longitudinal motion between the movable part 70 and the device body 60. When reaching a final assembly configuration as e.g. illustrated in any of the FIGS. 3-5 the spacer 80 can be detached from the add-on device 30 in step 204. For detaching the spacer 80 from the add-on device 30 the user may use the gripping portion 82 as a kind of a pull tab.

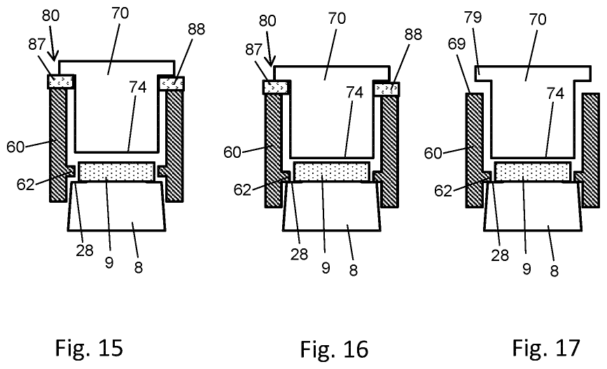

A typical scenario for mounting the add-on device 30 to the proximal end of the injection device 1 is further illustrated by the sequence of FIGS. 15-17. While assembling the add-on device 30 on the dose dial 8 of the injection device 1 there may be reached an intermediate configuration as illustrated in FIG. 15. There, the device body 60 of the add-on device has not yet reached a final assembly configuration on the dose dial 8 or trigger 9 of the injection device 1.

To reach a well-defined final assembly configuration as illustrated in FIG. 16, in which the abutment face 74 of the movable part 70 is in a well-defined distance or is even in abutment with the proximal face of the trigger 9 it may be necessary to push the device body 60 as illustrated in FIG. 15 further in distal direction onto the dose dial 8. The frictional engagement between an outside surface of the dose dial 8 and an inside of the sidewall 61 of the device body 60 may include the application of a distally directed force of substantial magnitude onto the device body 60 relative to the dose dial 8 so that the flange portion 62 of the device body 60 gets in abutment with the proximally facing end face 28 of the dose dial 8, thereby reaching the final assembly position.

For this, the user may be tempted to push onto the movable part 70. Since the spacer 80 is located between the movable part 70 and the device body 60 and since the spacer 80 substantially filles a gap between a proximal end face 69 of the device body 60 and a projection 79 of the movable part 70 any distally directed force effect applied onto the movable part 70 will transfer via the spacer 80 and its arm portions 87, 88 directly onto the sidewall 61 of the device body 60 until the final assembly configuration as illustrated in FIG. 16 has been reached. There, the flange portion 62 of the device body 60 is in longitudinal or axial abutment with the end face 28 of the dose dial 8.

Thereafter, the spacer 80 can be detached from the add-on device 30 and the movable part 70 becomes movable relative to the device body 60. Here, and after removal of the spacer 80 the movable part 70 is and remains in a well-defined position relative to the trigger 9 of the injection device 1. By way of the spacer 80 it can be ensured, that the movable part 70 is and remains in the predefined position or configuration relative to the device body 60 at least during the process of mounting the add-on device 30 to the injection device 1.

Otherwise, and without the spacer the movable part 70 may be subject to a longitudinal displacement relative to the device body 60, e.g. during mounting of the add-on device to 30 to the injection device 1, which relative movement may lead to an unintended activation or use of the add-on device 30. With the spacer 80 attached to the add-on device 30 it can be ensured, that the predefined position of orientation of the movable part 70 relative to the device body 60 can be maintained during assembly of the add-on device 30 to the injection device 1.

With the example as illustrated in FIGS. 15-17 the add-on device 30 may be void of a return element 65. Here, the movable part 70 may only return into a proximal end position under the action or effect of the returning trigger 9, which in turn is typically biased in proximal direction 3 by a trigger spring (not illustrated). With such configurations, i.e, wherein the add-on device 30 is void of a return element 65 the correct assembly and positioning of e.g. the movable part 70 relative to the trigger 9 may of particular importance for a correct operation of the add-on device 30.

### REFERENCE NUMBERS

1 injection device
2 distal direction
3 proximal direction
4 dose incrementing direction
5 dose decrementing direction
6 body
7 cartridge holder
8 dose dial
9 trigger
10 housing
11 connector
12 needle assembly
13 injection needle
14 inner needle cap
15 outer needle cap
16 protective cap
18 piston
19 piston rod
20 drive mechanism
21 medicament container
22 barrel
23 seal
24 medicament
25 outlet
26 window
27 dial extension
28 end face
30 add-on device
34 electronic module
36 printed circuit board
38 transceiver
40 memory
42 clock
44 processor
46 power source
48 acceleration sensor
49 rotation sensor
50 position sensor
52 signal generator
60 device body
61 sidewall
62 flange portion
63 receptacle
64 receptacle
65 return element
66 projection
67 abutment face
68 scale
69 end face
69a recessed structure
70 movable part
71 stem
72 distal portion
73 proximal portion
73a abutment face
74 abutment face
75 cover
76 end face
77 sidewall
78 groove
79 projection
79a distal face
80 spacer
81 spacer body
82 gripping portion
83 engaging portion
84 receptacle
85 clasp
86 insert opening
87 arm portion
87a free end section
87 inside edge
88 arm portion
88a free end section
88b inside edge
89 sidewall
90 proximal face
91 distal face
92 gripping rib
93 inner edge
94 projection
95 through recess
110 hand
114 thumb
116 finger
117 finger

1-25. (canceled)

26. A spacer detachably fastenable to an add-on device of an injection device, the add-on device comprising a device body and a movable part, the movable part configured to move relative to the device body along a first direction, the spacer comprising:
a spacer body comprising an engaging portion to detachably fasten to at least one of the device body or the movable part,
wherein the spacer body is operable to impede a movement of the movable part relative to the device body along the first direction when the spacer body is fastened to the device body or the movable part.

27. The spacer according to claim 26, wherein the engaging portion comprises a clasp to at least partially embrace a sidewall of the movable part or to at least partially embrace a sidewall of the device body.

28. The spacer according to claim 27, wherein the clasp is configured to at least partially embrace the sidewall of the movable part and the sidewall of the device body.

29. The spacer according to claim 26, wherein the engaging portion comprises an insert opening to receive at least one of the movable part and the device body.

30. The spacer according to claim 26, wherein the spacer body is of planar or disc-like shape.

31. The spacer according to claim 26, wherein a thickness of at least one of the spacer body or the engaging portion is adapted to or conforms to a recessed structure provided on an outside surface of the add-on device.

32. The spacer according to claim 31, wherein a size of the recessed structure of the add-on device along the first direction matches with the thickness of the at least one of the spacer body or the engaging portion of the spacer body.

33. The spacer according to claim 31, wherein, when the spacer is fastened to or attached to the add-on device, at least one of the spacer body or the engaging portion at least partially occupies the recessed structure of the add-on device to impede or prevent a movement of the movable part relative to the device body.

34. The spacer according to claim 26, wherein the spacer body comprises a gripping portion projecting from the engaging portion.

35. The spacer according to claim 34, wherein the gripping portion comprises a gripping flange.

36. The spacer according to claim 34, wherein the gripping portion comprises a gripping tab that outwardly protrudes or radially outwardly extends from the engaging portion.

37. The spacer according to claim 34, wherein the gripping portion comprises at least one gripping rib.

38. The spacer according to claim 37, wherein the spacer body is of planar or disc-like shape, and the at least one gripping rib protrudes from a plane of the spacer body.

39. The spacer according to claim 26, wherein the spacer body comprises a proximal face and a distal face, wherein the distal face is configured to abut with a proximal face of the device body, and the proximal face of the spacer body is configured to abut with a distal face of the movable part.

40. An add-on device for detachably connecting to an injection device, the add-on device comprising:
a device body,
a movable part configured to move relative to the device body along a first direction, and
a spacer detachably fastenable to at least one of the device body or the movable part and configured to impede a movement of the movable part relative to the device body at least along the first direction, the spacer comprising:
a spacer body comprising an engaging portion to detachably fasten to at least one of the device body or the movable part,
wherein the spacer body is operable to impede a movement of the movable part relative to the device body along the first direction when fastened to the device body or the movable part.

41. The add-on device according to claim 40, wherein the device body comprises a receptacle configured to receive a dose dial of the injection device.

42. The add-on device according to claim 40, further comprising an electronic module arranged inside the device body or inside the movable part, wherein the electronic module comprises a processor, a memory, and a transceiver.

43. The add-on device according to claim 40, further comprising a recessed structure on an outside of a sidewall of the add-on device, wherein a size of the recessed structure along the first direction matches with a respective thickness of the spacer body of the spacer.

44. The add-on device according to claim 43, wherein the recessed structure on an outside of the add-on device is confined by a proximal face of the device body and a distal face of the movable part.

45. An injection device for setting and injecting of a dose of a medicament, the injection device comprising:
a housing to accommodate a medicament container,
a drive mechanism operably engageable with the medicament container for injecting of the dose of the medicament,
a dose dial operable by a user for setting of the dose,
a trigger depressible by the user for injecting the dose, and
an add-on device attachable to at least one of the dose dial or the trigger, the add-on device comprising:
a device body,
a movable part configured to move relative to the device body along a first direction, and
a spacer detachably fastenable to at least one of the device body or the movable part and configured to impede a movement of the movable part relative to the device body along the first direction, the spacer comprising:
a spacer body comprising an engaging portion to detachably fasten to at least one of the device body or the movable part,
wherein the spacer body is operable to impede a movement of the movable part relative to the device body along the first direction when fastened to the device body or the movable part.
